(19) United States
(12) Patent Application Publication
(10) Pub. No.: US 2026/0131119 A1
(43) Pub. Date: May 14, 2026

(54) DRUG ELUTING BALLOON WITH RADIAL PROJECTIONS

(71) Applicant: TERUMO MEDICAL CORPORATION, Somerset, NJ (US)

(72) Inventors: Sarah GETTINGER, West Chester, PA (US); Seiichi OTSUBO, Newark, DE (US); Kevin RIDGLEY, Newark, DE (US)

(73) Assignee: TERUMO MEDICAL CORPORATION, Somerset, NJ (US)

(21) Appl. No.: 19/388,552

(22) Filed: Nov. 13, 2025

Related U.S. Application Data
(60) Provisional application No. 63/720,462, filed on Nov. 14, 2024.

(51) Int. Cl. A61M 25/10

(52) U.S. Cl. CPC A61M 25/104; A61M 2025/105; A61M 2025/1052; A61M 2025/1081; A61M 2025/1084

(57) ABSTRACT

A balloon catheter device includes a catheter and a balloon assembly. The catheter defines a guidewire lumen and a drug lumen. The balloon assembly includes a balloon coupled to an outer surface of the catheter, a proximal retainer coupled to the catheter at a proximal end of the balloon, a distal retainer coupled to the catheter at a distal end of the balloon, and a plurality of restraints coupled between the proximal retainer and the distal retainer. The balloon is configured to receive a drug from the drug lumen. When the balloon is in an expanded state, the balloon has a plurality of restrained portions, each extending along a length of one of the restraints, and a plurality of radially projecting portions, each positioned between adjacent of the restrained portions. The radially projecting portions extend further radially outward from the catheter than the restrained portions.

### CROSS-REFERENCE TO RELATED PATENT APPLICATION

This application claims the benefit of and priority to U.S. Provisional Patent Application No. 63/720,462, filed Nov. 14, 2024, which is incorporated herein by reference in its entirety.

### TECHNICAL FIELD

The present application relates generally to drug eluting apparatuses for delivering a drug, such as a drug that includes an anti-proliferative agent, into a blood vessel. More specifically, the present application relates to drug eluting apparatuses that allow for delivery of a drug through a balloon with radially projecting portions, so as to deliver the drug into the vessel and to allow the drug to pool against the vessel in channels formed between the radially projecting portions of the balloon.

### BACKGROUND

Vascular diseases may result in stenosis and restenosis of vessels, in which the buildup of plaque on the walls of the vessels results in a narrowing of the vessels. Narrowing of a vessel may be detrimental to health if the vessel is not treated by reopening the vessel through a treatment such as angioplasty. During angioplasty, an expansion device, such as a balloon, is inserted into a vessel and advanced to a site of narrowing of the vessel. The device is then expanded in a radially outward direction against the vessel walls to expand the vessel. Such a device may also deliver a drug to the vessel, for example, through the balloon.

### SUMMARY

In one embodiment, a balloon catheter device includes a catheter and a balloon assembly coupled to a distal portion of the catheter. The catheter defines a guidewire lumen and a drug lumen. The balloon assembly includes a balloon having a proximal end and a distal end that are coupled to an outer surface of the catheter, a proximal retainer coupled to the outer surface of the catheter at the proximal end of the balloon, a distal retainer coupled to the outer surface of the catheter at the distal end of the balloon, and a plurality of restraints coupled between the proximal retainer and the distal retainer. The balloon is configured to receive a drug from the drug lumen and has an outer surface through which a plurality of openings extend. Each of the restraints extends along the balloon. When the balloon is in an expanded state, the balloon has a plurality of restrained portions, each extending along a length of one of the restraints, and a plurality of radially projecting portions, each positioned between adjacent of the restrained portions. The radially projecting portions extend further radially outward from the catheter than the restrained portions.

According to one aspect, the distal retainer includes a plurality of first arc-shaped portions, each of the first arc-shaped portions having a first arc length defined by adjacent of the restraints, the proximal retainer includes a plurality of second arc-shaped portions, each of the second arc-shaped portions having a second arc length defined by adjacent of the restraints, and the first arc length is equal to the second arc length.

According to one aspect, which is combinable with the above embodiment and aspects in any combination, the first arc-shaped portions of the distal retainer are circumferentially offset from the second arc-shaped portions of the proximal retainer.

According to one aspect, which is combinable with the above embodiment and aspects in any combination, the balloon catheter device further includes a second catheter and a blood-flow barrier deployable via the second catheter and configured to expand to block a vessel.

According to one aspect, which is combinable with the above embodiment and aspects in any combination, the second catheter defines an inflation lumen positioned within the catheter of the balloon catheter device and the blood-flow barrier has a proximal end and a distal end that are coupled to an outer surface of the second catheter. The blood-flow barrier is configured to receive an inflation fluid from the inflation lumen.

According to one aspect, which is combinable with the above embodiment and aspects in any combination, the blood-flow barrier includes an occluder device.

According to one aspect, which is combinable with the above embodiment and aspects in any combination, the balloon includes the restraints. The restraints are formed from first portions of the balloon that are thicker than second portions of the balloon forming the radially projecting portions.

According to one aspect, which is combinable with the above embodiment and aspects in any combination, the balloon includes the restraints. The restraints are formed from at least one of portions of the balloon bonded together or portions of the balloon bonded to the catheter.

According to one aspect, which is combinable with the above embodiment and aspects in any combination, the balloon assembly includes an external sheath that fits over the balloon, the external sheath including the restraints.

In another embodiment, a balloon catheter device includes a catheter and a balloon assembly coupled to a distal portion of the catheter. The catheter defines a guidewire lumen and a drug lumen. The balloon assembly includes a balloon having a proximal end and a distal end that are coupled to an outer surface of the catheter and a plurality of restraints extending between the proximal end of the balloon and an intermediate location between the proximal end and the distal end of the balloon. The balloon is configured to receive a drug from the drug lumen and has an outer surface through which a plurality of openings extend. Each of the restraints extend along the balloon. When the balloon is in an expanded state, the balloon has a first region between the proximal end and the intermediate location. The first region includes a plurality of restrained portions, each extending along a length of one of the restraints, and a plurality of radially projecting portions, each extending between adjacent of the restrained portions. The radially projecting portions extend further radially outward from the catheter than the restrained portions. When the balloon is in the expanded state, the balloon has a second region distal to the first region and configured to block a vessel.

According to one aspect, the balloon includes the restraints. The restraints are formed from first portions of the balloon that are thicker than second portions of the balloon forming the radially projecting portions.

According to one aspect, which is combinable with the above embodiment and aspects in any combination, the balloon includes the restraints. The restraints are formed from at least one of portions of the balloon bonded together or portions of the balloon bonded to the catheter.

According to one aspect, which is combinable with the above embodiment and aspects in any combination, balloon assembly includes an external sheath that fits over the balloon. The external sheath includes the restraints.

According to one aspect, which is combinable with the above embodiment and aspects in any combination, the balloon assembly includes a proximal retainer coupled to the outer surface of the catheter at the proximal end of the balloon and a second retainer coupled to the outer surface of the catheter at the intermediate location. The restraints are coupled between the proximal retainer and the second retainer.

According to one aspect, which is combinable with the above embodiment and aspects in any combination, the proximal retainer includes a plurality of first arc-shaped portions defined by adjacent of the restraints, the second retainer includes a plurality of second arc-shaped portions defined by adjacent of the restraints, and the first arc-shaped portions of the proximal retainer are circumferentially offset from the second arc-shaped portions of the second retainer.

In another embodiment, a balloon catheter device includes a catheter, a balloon assembly coupled to a distal portion of the catheter, and a blood-flow barrier. The balloon assembly includes a balloon having a proximal end and a distal end that are coupled to a first outer surface of the catheter. The balloon is configured to receive a drug from the drug lumen and has an outer surface through which a plurality of openings extend. The blood-flow barrier has a proximal end and a distal end that are coupled to a second outer surface of the catheter. The second outer surface is distal to the first outer surface. The blood-flow barrier is configured to receive an inflation fluid from the inflation lumen. When the balloon is in an expanded state, the balloon has a plurality of restrained portions and a plurality of radially projecting portions, each positioned between adjacent of the restrained portions, the radially projecting portions extending further radially outward from the catheter than the restrained portions. When the blood-flow barrier is in an expanded state, the blood-flow barrier is configured to block a vessel.

According to one aspect, the first outer surface is contiguous with the second outer surface.

According to one aspect, which is combinable with the above embodiment and aspects in any combination, the balloon includes a plurality of restraints extending between the proximal end of the balloon and the distal end of the balloon. Each of the restraints extend along one of the restrained portions of the balloon to restrain the restrained portions of the balloon. The restraints are formed from first portions of the balloon that are thicker than second portions of the balloon forming the radially projecting portions.

According to one aspect, which is combinable with the above embodiment and aspects in any combination, the balloon includes a plurality of restraints extending between the proximal end of the balloon and the distal end of the balloon. Each of the restraints extend along one of the restrained portions of the balloon to restrain the restrained portions of the balloon. The restraints are formed from at least one of portions of the balloon bonded together or portions of the balloon bonded to the catheter.

According to one aspect, which is combinable with the above embodiment and aspects in any combination, the balloon assembly includes an external sheath that fits over the balloon. The external sheath includes a plurality of restraints extending between the proximal end of the balloon and the distal end of the balloon. Each of the restraints extend along one of the restrained portions of the balloon to restrain the restrained portions of the balloon.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a perspective view of a balloon catheter device, according to various embodiments.

FIG. 2 is a perspective view of a balloon catheter device, according to various embodiments.

FIG. 3 is a perspective view of a balloon catheter device, according to various embodiments.

FIG. 4 shows a cross-sectional view of the balloon catheter device of FIG. 3 along plane A-A, according to various embodiments.

FIG. 5 shows a cross-sectional view of the balloon catheter device of FIG. 3 along plane B-B, according to various embodiments.

FIG. 6 shows a cross-sectional view of the balloon catheter device of FIG. 3 along plane C-C, according to various embodiments.

FIG. 7 shows a cross-sectional view of the balloon catheter device of FIG. 3 along plane A-A, according to various embodiments.

FIG. 8 shows a cross-sectional view of the balloon catheter device of FIG. 3 along plane B-B, according to various embodiments.

FIG. 9 shows a cross-sectional view of the balloon catheter device of FIG. 3 along plane C-C, according to various embodiments.

FIG. 10 is a perspective view of a balloon catheter device, according to various embodiments.

FIG. 11 shows cross-sectional views of the balloon catheter device of FIG. 3, taken along the plane D-D, according to various embodiments.

FIG. 12 shows a cross-sectional view of the balloon catheter device of FIG. 3, taken along the plane D-D, according to various embodiments.

FIG. 13 shows a cross-sectional view of the balloon catheter device of FIG. 3, taken along the plane D-D, according to various embodiments.

FIG. 14 shows a cross-sectional view of the balloon catheter device of FIG. 3, taken along the plane D-D, according to various embodiments.

FIG. 15 shows a cross-sectional view of the balloon catheter device of FIG. 3, taken along the plane D-D, according to various embodiments.

FIG. 16 is a perspective view of the distal end of the balloon catheter device shown in FIG. 10, taken along the plane E-E in FIG. 10.

FIG. 17 is a cross-sectional view of the balloon catheter device shown in FIG. 10, taken along the plane F-F in FIG. 10.

FIG. 18 is perspective view of the distal end of the balloon catheter device shown in FIG. 10, taken along the plane E-E in FIG. 10.

FIG. 19 is a perspective view of a balloon catheter device according to various embodiments.

FIG. 20 is a perspective view of a balloon catheter device according to various embodiments.

FIG. 21 shows a cross-sectional view of the balloon catheter device of FIG. 20, taken along plane G-G, according to various embodiments.

FIG. 22 shows a across-sectional view of the balloon catheter device of FIG. 20, taken along plane H-H, according to various embodiments.

FIG. 23 shows a cross-sectional view of the balloon catheter device of FIG. 20, taken along plane J-J, according to various embodiments.

FIG. 24 shows a cross-sectional view of the balloon catheter device of FIG. 20, taken along plane G-G, according to various embodiments.

FIG. 25 shows a cross-sectional view of the balloon catheter device of FIG. 20, taken along plane H-H, according to various embodiments.

FIG. 26 shows a cross-sectional view of the balloon catheter device of FIG. 20, taken along plane J-J, according to various embodiments.

FIG. 27 shows a cross-sectional view of the balloon catheter device of FIG. 20, taken along plane G-G, according to various embodiments.

FIG. 28 shows a cross-sectional view of the balloon catheter device of FIG. 20, taken along plane H-H, according to various embodiments.

FIG. 29 shows a cross-sectional view of the balloon catheter device of FIG. 20, taken along plane J-J, according to various embodiments.

FIG. 30 is a perspective view of a balloon catheter device according to various embodiments.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### I. General Overview of Balloon Catheter Devices

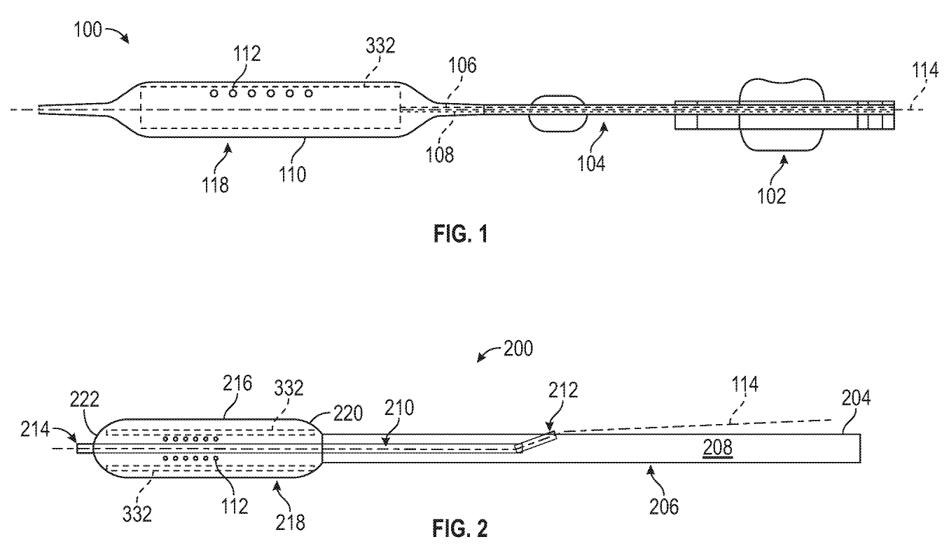

FIG. 1 shows a perspective view of a balloon catheter device 100. The balloon catheter device 100 is configured to be used in a procedure, such as angioplasty to treat stenosis or restenosis of a vessel. The balloon catheter device 100 may include a hub 102. The hub 102 allows a physician to control and guide the balloon catheter device 100 during the procedure. The hub 102 includes ports to receive an inflation fluid (e.g., saline, contrast, a saline-contrast mixture, etc.) or a drug (e.g., a drug that includes an anti-proliferative agent such as sirolimus, etc.). The balloon catheter device 100 includes a catheter 104 that is connected to the hub 102. The catheter 104 allows the balloon catheter device 100 to navigate to a position of interest within the patient for treatment. Once the balloon catheter device 100 is at the position of interest, the physician administers the drug and/or causes flow of the inflation fluid via the ports in the hub 102. The balloon catheter device 100 then enters an expanded state (e.g., inflated) at the position of interest as the drug and/or the inflation fluid fills the balloon catheter device 100. The pressure within the balloon catheter device 100 at its expanded state may be, for example, 0.5 to 18 atmosphere (at), 7 to 270 pounds per square inch (psi), etc. At such pressures, the balloon catheter device 100 of the present disclosure is configured to retain various target shapes as discussed in detail herein.

The catheter 104 includes a drug lumen 106. A lumen is a cavity or channel within a tube or tubular structure. A lumen provides a space through which the drug or the inflation fluid may flow. The drug lumen 106 is configured to receive and administer the drug. The drug lumen 106 may be contiguous with the ports in the hub 102 such that the drug flows from the ports and through the drug lumen 106. In some embodiments (e.g. when deployed with a blood-flow barrier, blocking balloon, etc.), the catheter 104 includes an inflation lumen 108 configured to receive the inflation fluid to inflate the balloon catheter device 100. As discussed herein, the catheter 104 may provide various structures to define the drug lumen 106 and/or the inflation lumen 108 (e.g., voids within a shaft, multiple independent tubes, multiple pipes defining separate flow annuluses, etc.).

The balloon catheter device 100 includes a balloon 110. The balloon 110 is configured to be attached (e.g., coupled, etc.) to a distal region of the catheter 104. The drug or inflation fluid is delivered to the balloon 110 and causes the balloon 110 to enter the expanded state. The balloon 110 has a plurality of openings 112 extending through the outer surface 118 such that when the balloon 110 is inflated, the drug is administered into a vessel through the openings 112. The balloon 110 includes a plurality of restraints 332 (e.g., restraining wires, restraining portions, restraining bands, etc.) which prevent the balloon 110 from inflating in all radial directions. For example, when the balloon 110 is inflated, the restraints 332 may restrain some portions of the balloon 110 while other portions of the balloon 110 expand outward until they press against the wall of the vessel. In this way, while some regions of the inflated balloon 110 expand to touch the vessel, other regions do not expand to touch the vessel and allow blood to continue flowing past the balloon 110. The openings 112 along the outer surface 118 of the balloon 110 may be directly adjacent to the walls of the vessel as the drug is released or they may be along regions of the outer surface 118 of the balloon 110 that are not directly adjacent to the walls of the vessel. In various embodiments, the openings 112 administer the drug into channels formed in the outer surface 118 of the balloon 110 while the balloon 110 is in the expanded state.

The catheter 104 and the balloon 110 (and optionally the hub 102) are configured to receive a guidewire 114. The particular guidewire 114 may vary (e.g., guidewires may vary in size, length, composition, coating, etc.). The guidewire 114 leads the balloon catheter device 100 until the balloon 110 is in place near the region of the vessel where treatment is desired. In some embodiments, the guidewire 114 extends through the hub 102 such that the balloon catheter device 100 is an “Over-the-Wire” (OTW) type device. In an OTW device, the guidewire 114 extends through the length of the balloon 110, through the entire length of the catheter 104, and through the hub 102.

FIG. 2 shows a perspective view of a balloon catheter device 200. As shown in FIG. 2, the balloon catheter device 200 is a “rapid-exchange” (RX) type device. In these embodiments, the balloon catheter device 200 is also configured to receive a guidewire 114. The balloon catheter device 200 has a distal end 214. The guidewire 114 is configured to extend from the distal end 214. However, the rapid exchange design allows the balloon catheter device 200 to use a shorter, and thus more manageable, length of the guidewire 114 than would be used in an OTW type device. The balloon catheter device 200 includes a catheter 204 configured to allow the guidewire 114 to be received in an RX manner. In various embodiments, the catheter 204 provides support for the balloon catheter device 200. For example, in some embodiments, the balloon 216 is coupled to the outer surface 206 of the catheter 204. The catheter 204 includes a drug lumen 208. The drug lumen 208 may be a hollow cavity extending through the length of the catheter 204, or may be defined by a separate, smaller drug tube located inside the outer surface 206 of the catheter 204, etc.

The balloon catheter device 200 includes a guidewire lumen 210. The guidewire lumen 210 is in an RX configuration. Unlike the drug lumen 208, the guidewire lumen 210 extends along a portion of a distal region of the catheter 204. The guidewire lumen 210 includes an RX port 212 which allows the guidewire 114 to exit from an opening running through the outer surface 206 of the catheter 204. In this way, the guidewire 114 extends along the outside of the catheter 204 the remaining proximal length such that the overall length of the guidewire 114 is substantially shorter than if the guidewire 114 extended through the entire length of the catheter 204 (e.g., as in an OTW type device). The guidewire lumen 210 is configured to terminate at the distal end 214 of the balloon catheter device 200. In some embodiments, the distal end 214 of the balloon catheter device 200 is defined solely by the guidewire lumen 210. In other embodiments, the outer surface 206 of the catheter 204 extends to the same length as the guidewire lumen 210 such that the distal end 214 is defined by the outer surface 206 of the catheter 204.

The balloon catheter device 200 includes a balloon 216. The balloon 216 has an outer surface 218. The outer surface 218 is configured to expand to abut against a wall of the vessel at certain portions. The outer surface 218 may compress plaque buildup on the wall of the vessel or otherwise expand the vessel. The outer surface 218 includes a plurality of openings 112 extending therethrough. When the drug inflates the balloon 216 via the drug lumen 208, the balloon 216 expands to partially abut the wall of the vessel; the drug then exits the balloon 216 via the openings 112 in the outer surface 218. The balloon 216 includes a plurality of restraints 332 that keep the balloon 216 in a target shape when the balloon 216 is in the expanded state. The balloon 216 includes a proximal end 220. The proximal end 220 is coupled to the outer surface 206 of the catheter 204 such that the balloon 216 is sealed at the proximal end 220. Similarly, the balloon 216 includes a distal end 222. The distal end 222 is coupled to the outer surface 206 of the catheter 204 such that the balloon 216 is sealed at the distal end 222. In other embodiments, the proximal end 220 and/or the distal end 222 are coupled to other structures connected to or adjacent to the outer surface 206 (e.g., retainers, endcaps, etc. secured to the catheter 204).

Regarding the terms “proximal” and “distal,” a component that is “proximal” is one that is closer to the end of the entire catheter device nearest the physician than its “distal” counterpart, which is closer to the end of the device furthest away from the physician. For example, a guidewire (e.g., the guidewire 114, etc.) may be placed with one end in the physician’s hand, its length extending into a patient’s vasculature, and an opposite end located in a particular vessel of the patient. The end of the guidewire in the physician’s hand (i.e., nearer the physician) is the proximal end. The end of the guidewire located in the patient’s vessel (i.e., further from the physician) is the distal end. One component may also be more proximal or more distal to another. For example, the hub 102 of the balloon catheter device 100 is proximal to the balloon 110. Likewise, when the balloon 110 is placed in a vessel, the balloon 110 is distal to the hub 102.

In typical drug-eluting balloon catheters, and in contrast to the balloon catheter device 100 and/or the balloon catheter device 200, the expanded state results in the balloon inflating such that the balloon fully blocks the vessel. When delivering a drug into the vessel using a drug eluting apparatus, it is desirable to deliver the drug through a balloon catheter device of the present disclosure which has a structure that defines channels in the balloon while the device is inflated/in the expanded state. In other words, rather than fully inflate the balloon to block the vessel and administer the drug directly against the vessel wall through openings in the balloon, radially projecting portions of the balloon inflate while other portions are restrained. By defining channels in the expanded state and administering the drug through openings in the balloon wall adjacent to the channels, the drug pools against the vessel wall. Allowing the drug to pool near the vessel wall increases the effectiveness of treatment. A drug that pools near the vessel wall remains at the area of interest for a longer duration, increasing exposure time between the drug and vessel wall rather than washing away downstream. Accordingly, the channels and corresponding pooling improves the efficiency of drug absorption and minimizes the amount of drug needed to achieve a remedial effect.

Further, the channels defined in the balloon’s outer surface allow blood to flow past the balloon while the drug is administered. Balloon catheters in the prior art block a vessel and prohibit blood flow while the balloon is in the expanded state and the drug is administered. Blocking the vessel and prohibiting blood flow limits the duration of the treatment because a blocked vessel increases the risk of blood clotting and other negative effects. Thus, by allowing blood flow to at least partially continue during the treatment process, the balloon catheter devices of the present disclosure increase the length of time that the drug can be administered without risking unwanted blood clotting or similar negative effects of fully blocking the vessel.

A blood-flow barrier may be used to block the vessel downstream from the channels of the balloon with radial projecting portions, thereby sealing the channels and allowing the drug to pool to an even greater extent against the vessel wall. In some embodiments, the blood-flow barrier is a blocking balloon configured to receive an inflation fluid via an inflation lumen. The blocking balloon may be deployed in a deflated state to a location within a vessel, then inflated with inflation fluid until the blocking balloon expands to block the vessel. In other embodiments, the blood-flow barrier is an occluder device (e.g., a PFO occluder, a cardioform septal occluder, a double-disk occluder, etc.), an umbrella-shaped blocking structure, or other similar structures that expand to block the vessel. The use of a blood-flow barrier in tandem with a balloon defining channels may also increase the duration of treatment. For example, the blood-flow barrier may be deflated or returned to a non-expanded state to allow blood flow to resume, while the balloon defining channels remains inflated and continues to administer the drug. In this way, the drug will pool against the area of interest in the vessel at much greater concentrations over longer periods of time when compared to balloon catheter devices present in the prior art.

Various embodiments of the balloon catheter devices described herein may provide one or more advantages over typical drug-eluting balloon catheters. For example, if no blood-flow barrier is present and treatment is administered with a balloon catheter device having channels in its outer surface while inflated, the device may administer drug into the vessel via a balloon with a smaller volume than a balloon that fully inflates and blocks a vessel. Because the balloon catheter device with channels does not fully expand, its volume is smaller than a balloon that radially expands in an unrestrained manner in all directions. Accordingly, less drug is used to inflate the balloon catheter device, and less drug is left inside the balloon (i.e., wasted) after the procedure is complete. As discussed above, the balloon catheter device allows for delivery of a drug that pools against the vessel rather than washing away due to blood flow, thereby improving the rate of drug absorption and wasting less drug. Additionally, without a blood-flow barrier, the balloon catheter device with channels allows for the delivery of the drug without fully blocking the vessel, allowing for a longer duration of treatment and decreased risk of blood clotting.

Other embodiments of balloon catheter devices disclosed herein achieve further benefits by being deployed in tandem with a blood-flow barrier. For example, when a balloon with channels and a blood-flow barrier are simultaneously deployed, the balloon catheter device may use an inflation fluid for the blood-flow barrier different from the drug used to inflate the balloon with channels. In this way, the vessel can be blocked using only inflation fluid and without using the drug, resulting in less waste of the drug that would otherwise remain in the blood-flow barrier after the drug is delivered to the vessel. In other embodiments, the blood-flow barrier is expanded to block the vessel without the use of an inflation fluid (e.g., an occluding device).

Various embodiments of the balloon catheter devices described herein may be used with a guiding catheter. A guiding catheter is inserted into a vessel before the balloon catheter device is inserted. The balloon catheter device may be inserted into and/or connected to the guiding catheter to facilitate entry of portions of the balloon catheter device into the vessel. Use of a guiding catheter protects the vessel from potential trauma that could be caused by the use of the balloon catheter devices without a guiding catheter. Further, the guiding catheter eases insertion of the balloon catheter device into the vessel and protects the balloon catheter device during insertion. The guiding catheter may be positioned at or near the target of drug elution.

### II. First Example Balloon Catheter Device

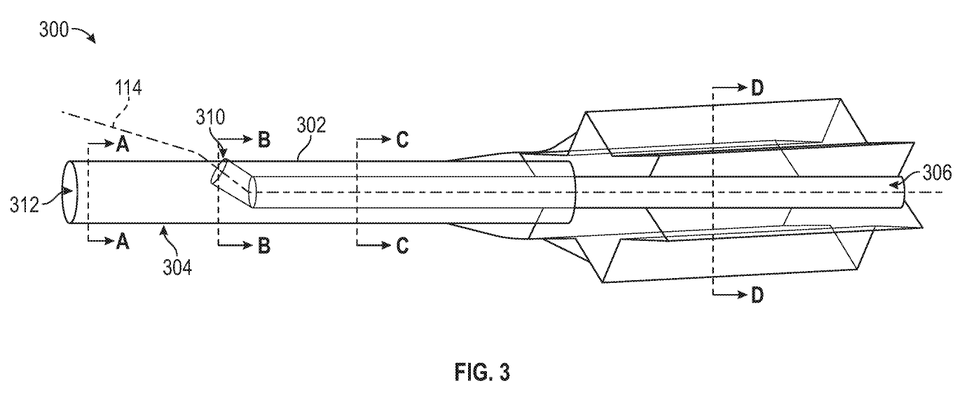

FIGS. 3-18 depict a balloon catheter device 300 for delivering a drug into a vessel. Turning to FIG. 3, the balloon catheter device 300 includes a catheter 302. The catheter 302 provides a structural base for other components of the balloon catheter device 300. The catheter 302 has an outer surface 304 onto which other components may be coupled. The catheter 302 also allows a physician to navigate and place the balloon catheter device 300 at a region of interest inside the vessel of a patient. The catheter 302 may be made of a fluorine polymer (e.g., polytetrafluoroethylene (PTFE)), ethylene/tetrafluoroethylene copolymer (ETFE), polyetheretherketone (PEEK), polyurethane, polyamide elastomer, polyester elastomer, polyurethane elastomer, polyimide, polycarbonate, polyetherimide, or other suitable materials.

The catheter 302 may have a length that is approximately in a range of 30-300 cm, for example, 50-200 cm, or 80-150 cm. As used herein, a range of X to Y includes X, Y, and values between X and Y. The catheter 302 may also have a cross-sectional shape that is a circle, an oval, a polygon, a rounded polygon, or other geometric shape. The catheter 302 may have a maximum cross-sectional width (e.g., a diameter where the cross-sectional shape is circular) that is approximately in a range of 0.5-10 mm, inclusive.

The outer surface 304 of the catheter 302 contains multiple lumens within its boundaries. For example, the catheter 302 includes a guidewire lumen 306 (e.g., pipe, cylinder, conduit, etc.). The balloon catheter device 300 includes the guidewire 114, which is configured to extend through the guidewire lumen 306. The guidewire 114 allows the balloon catheter device 300 to navigate through a patient’s vasculature without damaging or colliding with a wall of the vessel. The guidewire lumen 306 may have a cross-sectional shape that is a circle, an oval, a polygon, a rounded polygon, or other geometric shape. The guidewire lumen 306 may have a maximum cross-sectional width (e.g., a diameter where the cross-sectional shape is circular) that is approximately in a range of 0.1-1.5 mm, inclusive, and for example 0.3-1 mm. The guidewire lumen 306 may extend longitudinally through the catheter 302 along the entire length of the catheter 302. In this way, the balloon catheter device 300 may be an OTW type catheter device in which the guidewire 114 extends through and out of a port, inlet, opening, etc. at a proximal handle/hub attached to the catheter 302. In other embodiments and as shown in FIG. 3, the balloon catheter device 300 is an RX type catheter device, in which the guidewire 114 exits the catheter 302 at an RX port 310 located at a distal region of the catheter 302.

The catheter 302 includes a drug lumen 312 (e.g., cavity, space, tube, etc.). The drug lumen 312 may extend longitudinally through the catheter 302. The drug lumen 312 is configured to receive the drug (e.g., from a hub, drug port, drug inlet, handle, etc.) and deliver the same to the balloon catheter device 300. The drug lumen 312 may have a cross-sectional shape that is a circle, an oval, a polygon, a rounded polygon, or other geometric shape. The drug lumen 312 may have a maximum cross-sectional width (e.g., a diameter where the cross-sectional shape is circular) that is approximately in a range of 1 µm to 15 mm, inclusive, and, for example, 100 µm - 1 mm.

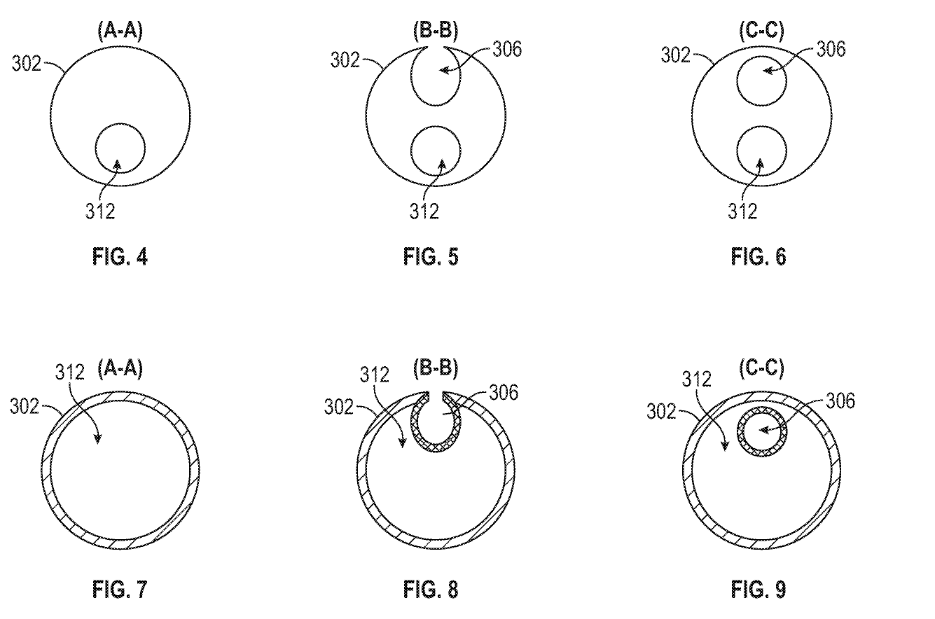

FIGS. 4-6 show an embodiment in which the catheter 302 is a solid shaft with voids therein defining the guidewire lumen 306 and the drug lumen 312. In FIG. 4, at plane A-A, a cross-section of the catheter 302 reveals the solid interior of the catheter 302 and the void of the drug lumen 312. In FIG. 5, a cross sectional view at B-B reveals the solid interior of the catheter 302, an entry for the guidewire lumen 306 at the top of the cross section, and the drug lumen 312. In FIG. 6, at C-C, a cross sectional view reveals the guidewire lumen 306 and the drug lumen 312 both running through the center of the solid catheter 302.

FIGS. 7-9 show one embodiment in which the catheter 302 is a hollow tube. Specifically, FIGS. 7-9 show a single specific embodiment in which the catheter 302 is constructed. FIGS. 7, 8, and 9 show different views from the specific single embodiment as viewed from three different cross sections (plane A-A, plane B-B, plane C-C) taken along the length of the catheter 302. In other words, FIG. 7 shows a cross sectional view at plane A-A of one embodiment. FIG. 8 shows a cross sectional view at plane B-B of the same embodiment. FIG. 9 shows a cross sectional view at plane C-C of the same embodiment. In FIG. 7, the interior of the catheter 302 forms the drug lumen 312, as shown in the cross-section of plane A-A. In FIG. 8, at B-B, a cross sectional view reveals the hollow interior of the catheter 302 forming the drug lumen 312. Additionally, a separate tube forms the guidewire lumen 306 at the top of the cross section of catheter 302. In FIG. 9, at C-C, a cross sectional view reveals the guidewire lumen 306 as a separate tube running longitudinally inside the hollow catheter 302, and the drug lumen 312 defined as the space between the outer wall of the catheter 302 and the inner tube of the guidewire lumen 306. Other variations and configurations of the guidewire lumen 306 and drug lumen 312 are possible. For example, the drug lumen 312 may itself be a separate hollow tube inside the catheter 302. Additionally, the guidewire lumen 306 may run within the entire length of the catheter 302 in an OTW type device.

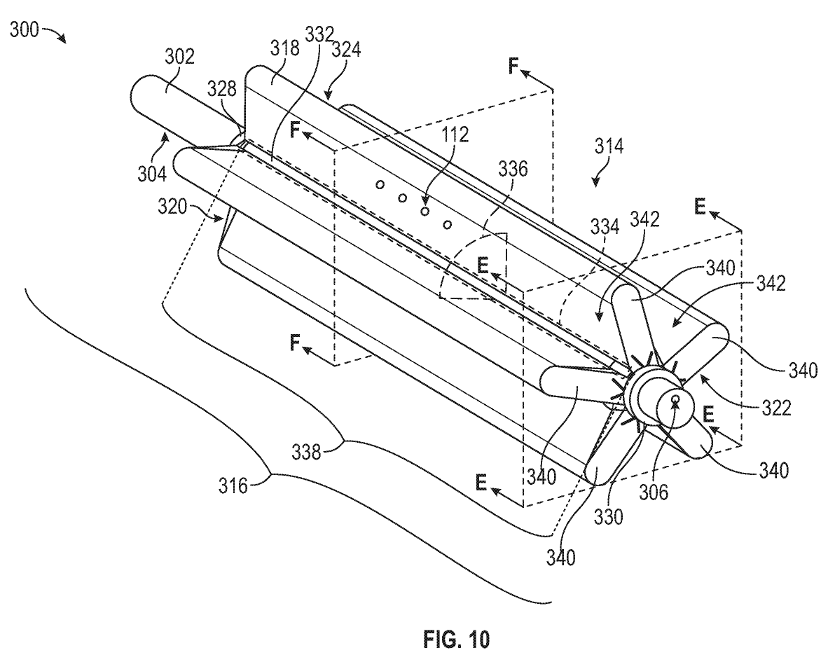

Turning to FIG. 10, a perspective view of an example balloon catheter device 300 according to the present disclosure is shown. The balloon catheter device 300 includes a balloon assembly 314 attached to a distal portion 316 of the catheter 302. The balloon assembly 314 is configured to administer the drug to the vessel. The balloon catheter device 300 administers the drug in an advantageous manner (e.g., in a manner that lessens drug volume waste, allows for increased contact/pooling between the drug and vessel, etc.). For example, when in the expanded state, channels are formed in the balloon catheter device 300. In the expanded state, the balloon catheter device 300 retains certain target shapes. While some portions of the balloon catheter device 300 expand radially outward and abut the walls of the vessel, other portions of the balloon catheter device 300 are restrained and form channels where blood flow continues and where the drug pools/accumulates against the walls of the vessel. Because the balloon catheter device 300 may not fully block the vessel, the duration of time that the drug can be administered is extended, allowing for a lengthier application of the drug during one procedure. Further, because some portions of the balloon catheter device 300 remain restrained even in the expanded state, the balloon catheter device 300 has a smaller volume than if it were allowed to fully expand (e.g., inflate to a cylindrical shape that fully blocked the vessel). With a smaller volume, the balloon catheter device 300 requires less drug to reach its expanded state, and less drug is wasted (e.g., by being left inside the device) when treatment is complete.

The balloon assembly 314 includes a balloon 318. The balloon 318 may be attached to the distal portion 316 of the catheter 302. For example, the balloon 318 may be attached to the distal portion 316 of the catheter 302 by being placed over the distal portion 316 of the catheter 302 and bonded with an adhesive, thermally fused together (e.g., heat welding, plastic welding), or connected by UV welding, ultrasonic welding, or other suitable connecting arrangements. The balloon 318 includes a proximal end 320 and a distal end 322. More specifically, the balloon 318 is connected to the catheter 302 at the proximal end 320 and the distal end 322. The balloon 318 includes an outer surface 324. The balloon 318 is configured to enter an expanded state upon receiving the drug, wherein channels form between the outer surface 324 and the wall of the vessel. In this way, the drug is more effectively administered by pooling in the channels between the outer surface 324 and the vessel. The balloon 318 may have a length that is approximately in a range of 10-400 mm. The balloon 318 may also have a width that is approximately in a range of 0.1-15 mm at its widest point. The balloon 318 may be made of polyamide, polyester, polyurethane, polyamide elastomer, polyester elastomer, polyurethane elastomer, or other suitable materials.

The outer surface 324 includes a plurality of openings 112. The openings 112 extend through the outer surface 324 and allow the drug inside the balloon 318 to flow out of the balloon 318 and into the vessel. The drug may pool, gather, and/or accumulate in channels formed between the outer surface 324 and the vessel while the balloon 318 is in an expanded state. The openings 112 may be circular, ovular, or another geometric shape, and may be symmetrically located around the outer surface 324, may be uniformly spaced along the length of the balloon 318, may be located at various locations on the outer surface 324, or may be disposed in other suitable arrangements along the balloon 318 to sufficiently administer the drug.

The balloon assembly 314 includes a proximal retainer 328. The proximal retainer 328 may attached to the outer surface 304 of the catheter 302 at or near the proximal end 320 of the balloon 318. The proximal retainer 328 may be made of plastic or other suitable materials, and may be attached to the outer surface 304 of the catheter 302 by being bonded with an adhesive, thermally fused to the catheter 302, connected with ultrasonic welding, UV welding, or with other suitable connections. The balloon assembly 314 includes a distal retainer 330 attached to the outer surface 304 of the catheter 302 at or near the distal end 322 of the balloon 318. The distal retainer 330 may be made of similar materials to and attached in similar methods as the proximal retainer 328. The proximal retainer 328 and distal retainer 330 provide boundary surfaces that assist in forming the channels in the balloon 318 while the balloon 318 is in the expanded state. In various embodiments, the proximal retainer 328 is identical to the distal retainer 330.

The balloon assembly 314 includes a plurality of restraints 332 (e.g., restraining bands, restraining portions, restraining elements, restraining wires, etc.). The restraints 332 are fastened at their ends such that the length of the restraints 332 are configured to only move radially outward a distance smaller than the distance that the balloon 318 would expand radially outward. In other words, portions of the balloon 318 expand radially outward past the restraints 332 when the balloon 318 is in an expanded state such that channels are formed along the outer surface 324 (e.g., by the restraints 332, etc.). Each of the restraints 332 may be a wire made of various materials (e.g., platinum, platinum/iridium, platinum/tungsten, stainless steel alloys, alloys of nickel and titanium, nitinol, plastic-coated alloys, etc.). The restraints 332 may be smooth, flat, braided, or coated to ensure sufficient durability and rigidity to restrain portions of the balloon 318 such that the balloon 318 retains a target shape when in the expanded state. In some embodiments, the restraints 332 may be made of nylon and polyethylene terephthalate (PET), suture thread, or like materials that have sufficient durability, rigidity, flexibility, and provide for visibility under fluoroscopy. In other embodiments, the restraints 332 may be made of the same material as the balloon 318 and/or the balloon 318 may include the restraints 332. For example, the balloon 318 may include the restraints 332 as portions of the balloon 318 having thicker widths than other portions of the balloon 318. In this this way, expanding the balloon 318 would result in a “wire” or band of material restraining certain portions of the balloon 318 from fully inflating. In other embodiments, the restraints 332 may be portions of the balloon 318 that secured in place and unable to expand while other portions are left unsecured. For example, the restraints 332 may be portions of the balloon 318 bonded with an adhesive, thermally fused together (e.g., heat welding, plastic welding), or connected by UV welding, ultrasonic welding, etc. to an internal section of the balloon 318 or a surface of the catheter 302 inside the balloon 318. Such bonding may result in the restraints 332 of the balloon 318 that are fixed and do not expand radially outward when the balloon 318 is inflated. In further embodiments, the restraints 332 may be included as part of a sheath (e.g., an external sheath, etc.) or casing made of the same material as the balloon 318, the materials described above, or like materials may be displaced over the balloon 318. In this way, when the balloon 318 inflates, the sheath or casing covers (e.g., fits over, etc.) the balloon 318 and the restraints 332 of the sheath or casing restrain some portions of the balloon 318 and allow other portions of the balloon 318 to fully expand. For a given balloon assembly 314, the restraints 332 may be made of any combination or variation of the above configurations, as well as like configurations. The target shape of the balloon 318 may be a star, a four-armed cross section, a three-armed cross section, a six-armed cross section, a spiraling three-armed cross section, etc. The restraints 332 may be coated to provide hydrophilic and/or lubricious qualities. The restraints 332 may have a cross-sectional shape that is a circle, an oval, a polygon, a rounded polygon, or other geometric shape. In some embodiments, the restraints 332 may have a cross-sectional width (e.g., a diameter where the cross-sectional shape is circular) that is approximately in a range of 0.1 mm to 5 mm.

Each of the restraints 332 is coupled to the proximal retainer 328 at one end and the distal retainer 330 at the other end. In this way, each of the restraints 332 is configured to restrain a corresponding portion 334 of the balloon 318 beneath the respective restraint 332. When the balloon 318 is in the expanded state, the restraints 332 keep corresponding portions 334 of the balloon 318 held down beneath each respective restraint 332 such that each corresponding portion 334 cannot fully expand radially outward. Accordingly, when the balloon 318 is in the expanded state, the restraints 332 and the corresponding portions 334 define a plurality of restrained portions 336 of the balloon 318. In other words, each respective restraint 332 and its corresponding portion 334 create a respective restrained portion 336 when the balloon 318 is in the expanded state. The restrained portions 336 have the effect of creating channels along at least a portion of the length of the balloon 318 for the drug to pool and/or accumulate near the wall of the vessel. The restrained portions 336 extend along a length 338 of a respective one of the restraints 332. When the balloon 318 is in the expanded state, the restraints 332 and corresponding restrained portions 336 create a plurality of radially projecting portions 340. The portions of the balloon 318 not restrained beneath the restraints 332 are able to radially expand outward as they inflate. This radial expansion creates the radially projecting portions 340 with each of the radially projecting portions 340 located between adjacent of the restrained portions 336.

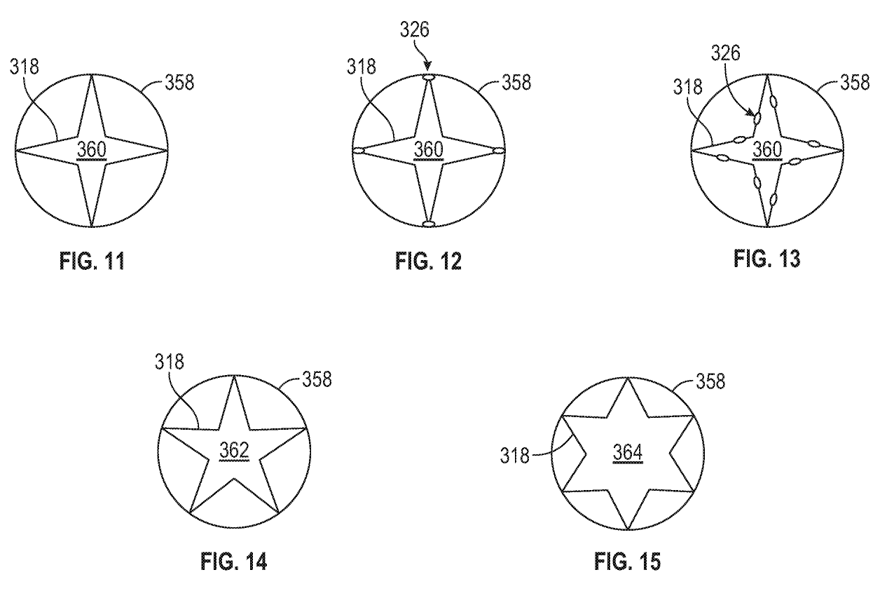

FIGS. 11-15 show cross-sectional views of a balloon catheter device 300 according to various embodiments, taken along the plane D-D in FIG. 3. In FIGS. 11-15, the balloon catheter device 300 is in the expanded state inside a vessel 358. In FIG. 11, the balloon catheter device 300 has, for example, a four-armed cross section 360 when in the expanded state. The four-armed cross section 360 of the balloon 318 may result from an expanded state of a balloon catheter device 300 having four restraints 332 in the balloon assembly 314. The four-armed cross section 360 may be many shapes. For example, the four-armed cross section 360 may abut the vessel 358 at pointed ends (as shown), rounded ends, squared-off ends, etc. The angle between each arm may also vary (e.g., 20 degrees, 30 degrees, etc.). FIG. 12 shows that the openings 326 may be located at various positions along the outer surface 324 of the balloon 318 that directly contact the vessel, such as at the radially outermost portions of the balloon 318. FIG. 13 shows that the openings 326 may be along various regions of the outer surface 324 of the balloon 318 that do not directly contact the vessel. In FIG. 14, the balloon catheter device 300 has, for example, a five-armed cross section 362 when in the expanded state. The five-armed cross section 362 of the balloon 318 results from an expanded state of a balloon catheter device 300 having five restraints 332 in the balloon assembly 314. FIG. 15 shows that the balloon catheter device 300 has, for example, a six-armed cross section 364 when in the expanded state. The six-armed cross section 364 of the balloon 318 results from an expanded state of a balloon catheter device 300 having six restraints 332 in the balloon assembly 314. The cross section of the balloon catheter device 300 may vary in other manners (e.g., have more or less arms, have varying angles between arms, have varying contours of the arms as the balloon 318 radially expands, etc.).

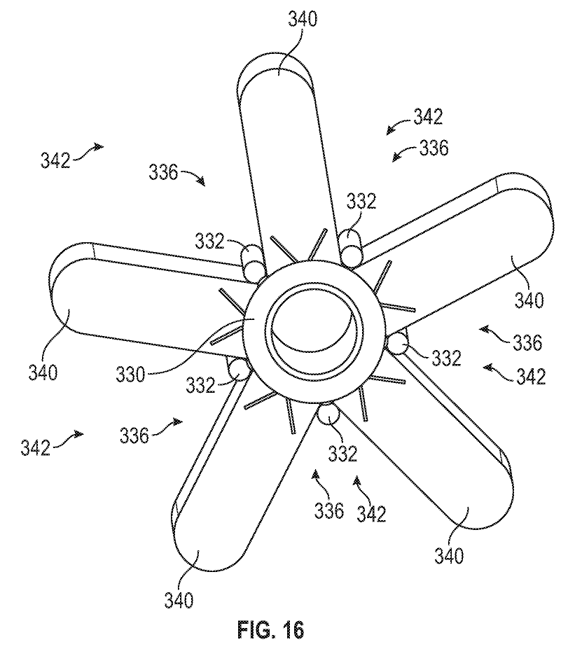

FIG. 16 shows a perspective cross-sectional view of the distal end of the balloon catheter device 300 of FIG. 10, taken along plane E-E in FIG. 10. The balloon assembly 314 includes five of the restraints 332, which create five of the restrained portions 336 when the balloon catheter device 300 is in the expanded state. In the expanded state, the balloon catheter device 300 also has five of the radially projecting portions 340 with each of the radially projecting portions 340 located between adjacent of the restrained portions 336.

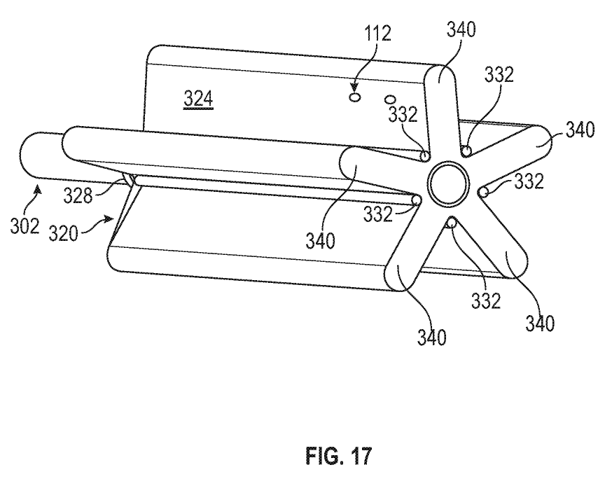

FIG. 17 shows a perspective cross-sectional view of the balloon catheter device 300 in FIG. 10, taken along the plane F-F in FIG. 10. As shown in FIG. 17, the radially projecting portions 340 may be configured to take on various shapes when in an expanded state. For example, in FIG. 17, the radially projecting portions 340 are rounded at their radially outermost regions and have rectangular longitudinal regions extending radially outward. In other embodiments, the radially projecting portions 340 may be pointed, squared off, etc. at their radially outmost regions. Further, the longitudinal regions of the radially projecting portions 340 may be oblong, tapered, or other geometric shapes at various angles of incline.

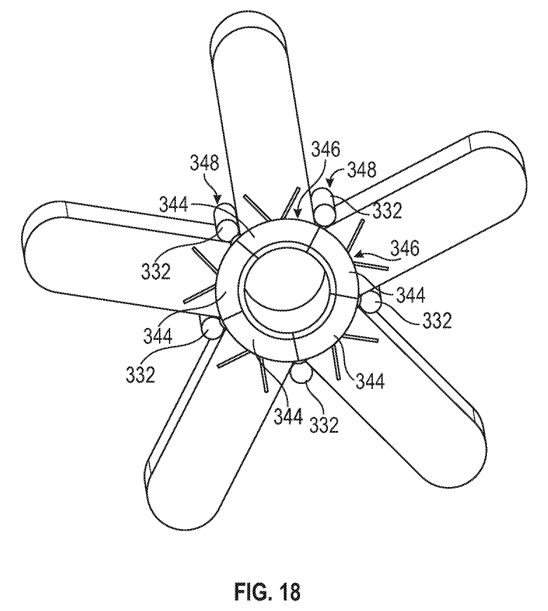

FIG. 18 shows a perspective cross-sectional view of the distal end of the balloon catheter device 300 shown in FIG. 10, taken along the plane E-E in FIG. 10. In various embodiments, the restraints 332 defines a plurality of arc-shaped portions 344 of the distal retainer 330. For example, five of the arc shaped portions 344 of the distal retainer 330 are shown in FIG. 18. In some embodiments, the distal retainer 330 may be a circular ring attached to the outer surface 304 of the catheter 302. Ends of the restraints 332 may attach to the distal retainer 330. As shown in FIG. 18, a first arc length 346 of the distal retainer 330 may be defined as the distance along the perimeter of the distal retainer 330 between adjacent of the restraints 332. The arc shaped portion 344 may be defined as the portion of the distal retainer 330 extending radially inward from the first arc length 346 of the distal retainer 330. In this way, a plurality of arc shaped portions 344 may be defined as the portions of the distal retainer 330, each portion extending radially inward from a respective first arc length 346.

Similarly, the restraints 332 may define a plurality of arc-shaped portions of the proximal retainer 328. The proximal retainer 328 may also be a circular ring attached to the outer surface 304 of the catheter 302. Ends of the restraints 332 may attach to the proximal retainer 328. A second arc length of the arc-shaped portions of the proximal retainer 328 may be defined as the distance along the perimeter of the proximal retainer 328 between adjacent of the restraints 332. An arc shaped portion of the proximal retainer 328 may be defined as the portion of the proximal retainer 328 extending radially inward from the second arc length of the arc-shaped portions of the proximal retainer 328. In this way, a plurality of arc shaped portions of the proximal retainer 328 may be defined as the portions of the proximal retainer 328, each portion extending radially inward from a respective second arc length of the arc-shaped portions of the proximal retainer 328.

In various embodiments, the first arc length 346 is equal to the second arc length of the arc-shaped portions of the proximal retainer 328 (e.g., the restraints 332 extend parallel to one another and are attached at equidistant intervals at their ends to the proximal retainer 328 and distal retainer 330, the restraints 332 are attached at equidistant intervals at their ends to the proximal retainer 328 and distal retainer 330 but extend in a spiraling manner between the proximal retainer 328 and distal retainer 330, the restraints 332 are attached at equidistant intervals at their ends to the proximal retainer 328 and distal retainer 330 and cross over one another as they extend between the proximal retainer 328 and distal retainer 330). By varying the locations on the proximal retainer 328 and distal retainer 330 to which each end of a respective restraint 332 attaches (e.g., at different locations around a longitudinal axis of the balloon catheter device 300, etc.), differing shapes and depths of channels may be formed on the outer surface 324 of the balloon 318 when it is in the expanded state. For example, when the arc-shaped portions 344 of the distal retainer 330 are circumferentially offset from the arc-shaped portions of the proximal retainer 328 (e.g., around a central axis of the balloon catheter device 300, etc.), the restraints 332 may extend at least partially in a circumferential direction around the balloon catheter device 300.

### III. Second Example Balloon Catheter Device

FIG. 19 shows a balloon catheter device 400 for delivering the drug into a vessel. In addition to the components discussed above in the previous embodiments, the balloon catheter device 400 includes a blocking balloon assembly 402 attached to a distal portion 404 of a catheter 406. The blocking balloon assembly 402 includes a balloon 408.

The blocking balloon assembly 402 has a proximal end 410 and a distal end 412 that are attached to an outer surface 414 of the catheter 406. In other embodiments, the distal end 412 of the blocking balloon assembly 402 may be attached to an endcap, attached to a retainer, attached directly to the outer surface 414 of the catheter 406, or may be connected to the catheter 406 in other suitable manners. In various configurations, portions of balloon 408 of the blocking balloon assembly 402 at the proximal end 410 and/or distal end 412 are configured to expand to block the vessel.

For example, in FIG. 19, the blocking balloon assembly 402 has a proximal retainer 416 attached to the outer surface 414 of the catheter 406 at or near the proximal end 410 of the blocking balloon assembly 402. The blocking balloon assembly 402 also has a second retainer 418 attached to the outer surface 414 of the catheter 406 at a location (e.g., an intermediate location, etc.) distal to the proximal retainer 416 and between the proximal end 410 of the blocking balloon assembly 402 and the distal end 412 of the blocking balloon assembly 402. For example, the second retainer 418 may be located at a midpoint along the length of the blocking balloon assembly 402 such that the second retainer 418 is distal to the proximal retainer 416. In some embodiments, the second retainer 418 divides or separates the balloon 408 into two regions. In such a configuration, the proximal retainer 416 and the second retainer 418 define a first region 420 of the blocking balloon assembly 402 between the proximal retainer 416 and the second retainer 418. Similarly, the second retainer 418 and the distal end 412 of the blocking balloon assembly 402 define a second region 422 of the blocking balloon assembly 402 between the second retainer 418 and the distal end 412 of the blocking balloon assembly 402.

The blocking balloon assembly 402 includes the restraints 332 coupled to the proximal retainer 416 and the second retainer 418. Each restraint 332 is configured to restrain a corresponding portion 426 of the first region 420 of the blocking balloon assembly 402 beneath each restraint 332 when the blocking balloon assembly 402 is in an expanded state. In an expanded state where the blocking balloon assembly 402 receives the drug through the drug lumen 312, the first region 420 of the blocking balloon assembly 402 forms a plurality of restrained portions 428 and a plurality of radially projecting portions 430, with each of the restrained portions 428 extending between adjacent of the restrained portions 428.

Additionally, when the blocking balloon assembly 402 is in the expanded state, the second region 422 of the blocking balloon assembly 402 is not restrained by any of the restraints 332 and thus the second region 422 may fully inflate to block the vessel. The first region 420 (e.g., the portion of the balloon 408 that is restrained and forms channels between the balloon and the vessel) and the second region 422 (e.g., the portion of the balloon 408 that is unrestrained and expands to block the vessel) may be connected at a boundary 409. For example, the boundary may be a seam, flap, joined region of the balloon, or other like structure.

### IV. Third Example Balloon Catheter Device

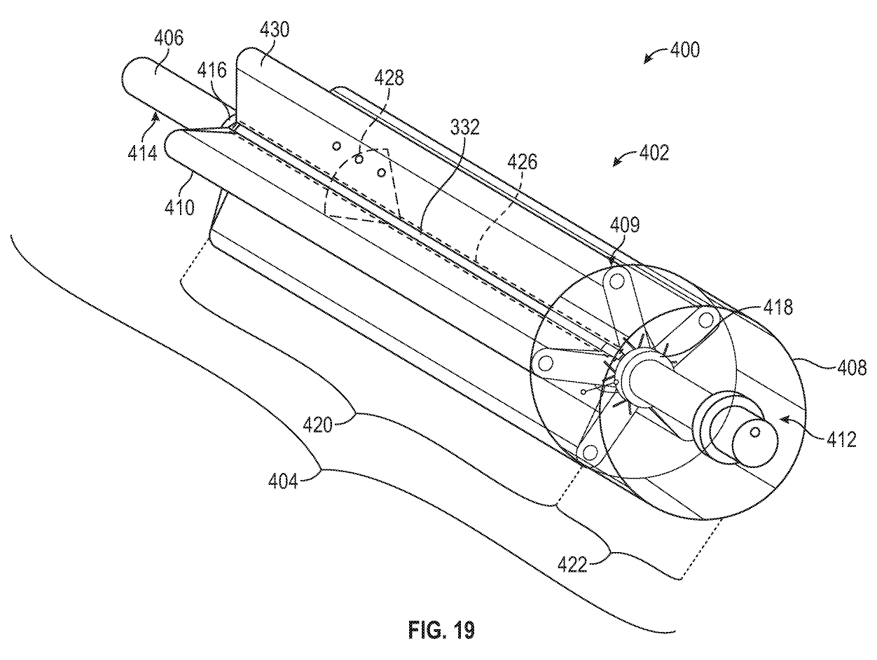

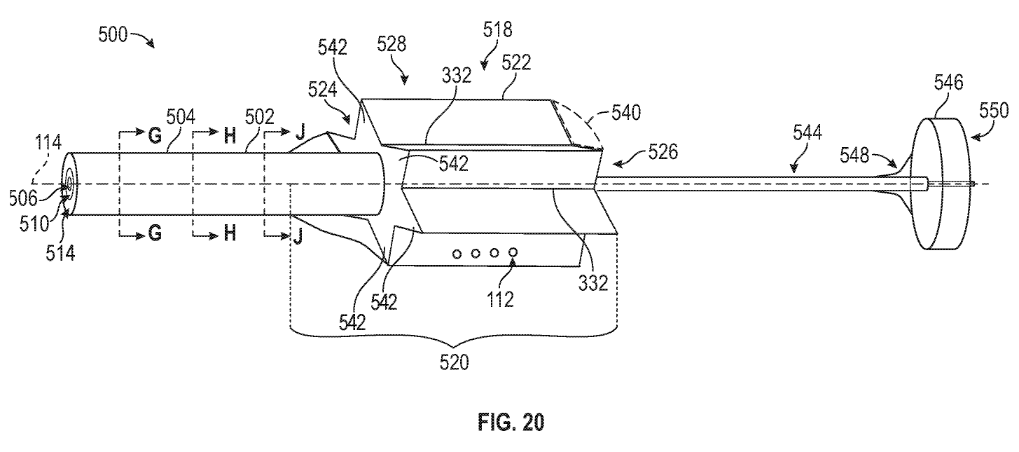

FIG. 20 shows a balloon catheter device 500 for delivering the drug into a vessel. The balloon catheter device 500 includes a catheter 502. In various embodiments, the catheter 502 includes a first outer surface 504 and a second outer surface 544 on which to couple the balloon catheter device 500. The first outer surface 504 of the catheter 502 contains various lumens within its boundaries. In some embodiments, the catheter 502 may include various lumens that extend to the same length. In other embodiments, the catheter 502 may include various lumens that extend to different lengths.

For example, the catheter 502 includes a guidewire lumen 506 that may extend further than any other lumen. The catheter 502 includes an inflation lumen 510 and a drug lumen 514. The inflation lumen 510 and the drug lumen 514 may be independent of one another such that the length of the inflation lumen 510 may increase or decrease without affecting the length of the drug lumen 514, or vice versa. For example, a physician may be able to deploy the inflation lumen 510 at a distance of 60 cm, deploy the drug lumen 514 at a distance of 50 cm, and extend the inflation lumen 510 to a distance of 70 cm without moving the drug lumen 514.

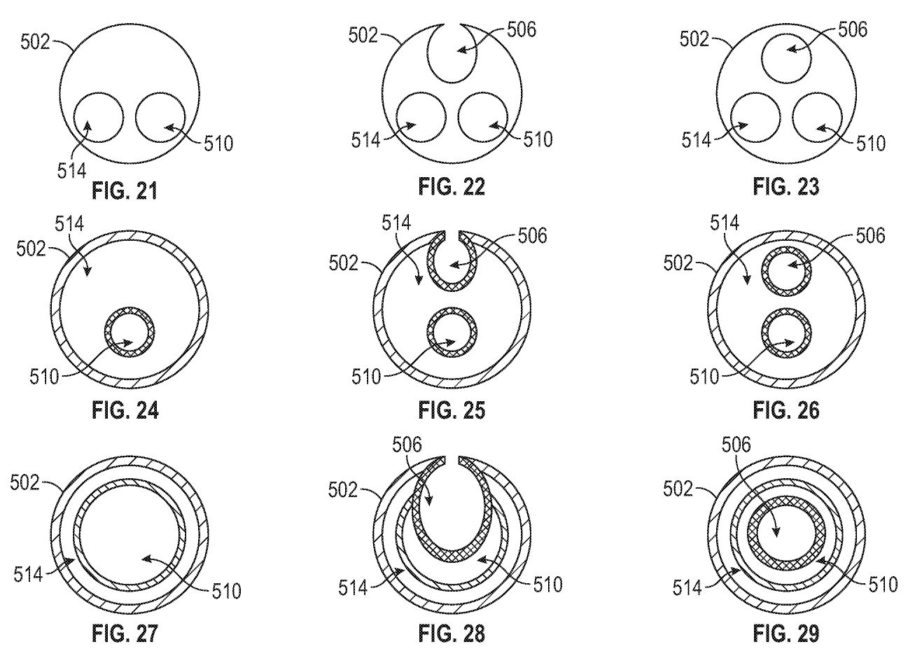

The catheter 502, guidewire lumen 506, inflation lumen 510, and drug lumen 514 may be disposed in various configurations as shown in FIGS. 21-29. FIGS. 21-29 show cross-sectional views of the balloon catheter device 500 shown in FIG. 20, wherein the catheter 502 is solid and contains voids that define the guidewire lumen 506, inflation lumen 510, and drug lumen 514. In FIG. 21, a cross sectional view taken at plane G-G shows the catheter 502 as a solid shaft with voids therein defining the inflation lumen 510 and the drug lumen 514. In FIG. 22, a cross sectional view taken at plane H-H reveals the solid interior of the catheter 502 and voids forming the drug lumen 514 and inflation lumen 510. Additionally, a void forming a guidewire lumen 506 (here, showing an RX configuration) is present at the top of the catheter 502. In FIG. 23, a cross-sectional view at plane J-J reveals the solid interior of the catheter 502, the guidewire lumen 506 at the top of the cross section, and the inflation lumen 510 and drug lumen 514 below the guidewire lumen 506 running through the center of the solid catheter 502.

As shown in FIGS. 24-26, the catheter 502 may be a hollow tube. In FIG. 24, a cross sectional view at plane G-G shows that the interior of the catheter 502 may form the drug lumen 514 and a separate tube may form the inflation lumen 510. In FIG. 25, a cross-sectional view at plane H-H reveals the hollow interior of the catheter 502 forming the drug lumen 514, a separate tube forming the guidewire lumen 506 at the top of the cross section, and a separate tube forming the inflation lumen 510. In FIG. 26, a cross-sectional view at plane J-J reveals the guidewire lumen 506 within a separate tube running longitudinally inside the hollow catheter 502, the inflation lumen 510 within a separate tube, and the drug lumen 514 defined as the space between the outer wall of the catheter 502 and the inner tubes of the guidewire lumen 506 and inflation lumen 510.

As shown in FIGS. 27-29, the drug lumen 514, inflation lumen 510, and guidewire lumen 506 may each be within separate sections of tubing. In FIG. 27, at plane G-G, the catheter 502 is a hollow tube and the inflation lumen 510 is within a separate hollow tube. The drug lumen 514 is defined as the hollow space between the tubes of the catheter 502 and the inflation lumen 510. In FIG. 28, at plane H-H, an additional hollow tube enters the top of the catheter 502 and contains the guidewire lumen 506. The inflation lumen 510 is defined as the space between the inner tube and the tube defining the guidewire lumen 506. The drug lumen 514 is the space between the catheter 502, the tube of the guidewire lumen 506, and the tube of the inflation lumen 510. Finally, in FIG. 29, at plane J-J, the guidewire lumen 506 is contained within a central hollow tube, the inflation lumen 510 is the space between the central hollow tube and a second hollow tube, and the drug lumen 514 is the space between the second hollow tube and the catheter 502. Other variations and configurations of the guidewire lumen 506, inflation lumen 510, and drug lumen 514 are possible. For example, the drug lumen 514 may itself be a separate hollow tube inside the catheter 502. Additionally, the guidewire lumen 506 may run along the entire length of the catheter 502 in an “over-the-wire” type device.

Turning back to FIG. 20, the balloon catheter device 500 includes a balloon assembly 518 attached to a distal portion 520 of the catheter 502. In some embodiments, the balloon assembly 518 is attached to the first outer surface 504 of the catheter 502. The balloon assembly 518 includes a balloon 522. The balloon 522 is configured to be attached to the distal portion 520 of the catheter 502, and specifically to be coupled to the first outer surface 504. The balloon 522 includes include a proximal end 524 and a distal end 526. More specifically, the balloon 522 may be connected to the catheter 502 at the proximal end 524 of the balloon 522 and the distal end 526 of the balloon 522. The balloon 522 includes an outer surface 528. The outer surface 528 includes a plurality of openings 112 that extend through the outer surface 528 and allow the drug to flow out of the balloon 522 and into the vessel. The balloon assembly 518 includes a proximal retainer (e.g., a proximal retainer 328 as shown in FIG. 10) attached to the catheter 502 at or near the proximal end 524 of the balloon 522. The balloon assembly 518 includes a distal retainer (e.g., a distal retainer 330 as shown in FIG. 10) attached to the catheter 502 at or near the distal end 526 of the balloon 522.

The balloon assembly 518 includes the restraints 332. Each of the restraints 332 is coupled to the proximal retainer of the balloon assembly 518 at one end and the distal retainer of the balloon assembly 518 at the other end. In this way, each of the restraints 332 may be configured to restrain a corresponding portion 538 of the balloon 522 beneath the respective restraint 332. When the balloon 522 is in the expanded state, each of the restraints 332 keeps its corresponding portion 538 of the balloon 522 held down beneath each respective restraint 332 such that each corresponding portion 538 cannot fully expand radially outward. When the balloon 522 is in the expanded state, the restraints 332 and corresponding plurality of restrained portions 540 also create a plurality of radially projecting portions 542. The portions of the balloon 522 not restrained beneath the restraints 332 are able to radially expand outward as they inflate.

The balloon catheter device 500 includes a blood-flow barrier 546. The blood-flow barrier 546 is configured to receive the inflation fluid from the inflation lumen 510. When the blood-flow barrier 546 receives the inflation fluid (or is otherwise deployed to enter an expanded state in the case of occluders and devices that do not require inflation fluid), the blood-flow barrier 546 enters an expanded state, wherein the blood-flow barrier 546 blocks the vessel. When the blood-flow barrier 546 is in the expanded state the blood-flow barrier may form an umbrella shape. The blood-flow barrier 546 includes a proximal end 548 and a distal end 550. The proximal end 548 and the distal end 550 are attached to the second outer surface 544. The second outer surface 544 can be distal to the first outer surface 504 of the catheter 502, such that the blood-flow barrier 546 is located downstream of the balloon assembly 518. For example, as shown in FIG. 20, the blood-flow barrier 546 is deployed distal to the balloon assembly 518, and the second outer surface 544 extends beyond the balloon assembly 518 such that the second outer surface 544 is distal to the balloon assembly 518. In other configurations, the second outer surface 544 can be located within the first outer surface 504 of the catheter 502. For example, if the blood-flow barrier 546 is guided towards the balloon assembly 518 such that the blood-flow barrier 546 abuts the balloon assembly, the second outer surface 544 is located within the balloon assembly 518 rather than distal to the balloon assembly 518. In some embodiments, like in FIG. 20, the second outer surface 544 of the catheter 502 may not be contiguous with the first outer surface 504. In such embodiments, the blood-flow barrier 546 is configured to move closer to or farther downstream from the balloon assembly 518. For example, the second outer surface 544 of the catheter 502 may be located on the exterior of the inflation lumen 510. The inflation lumen 510 may be configured to be pulled or translated in a proximal direction, thus pulling the blood-flow barrier 546 closer to the balloon assembly 518.

In other embodiments, the blood-flow barrier 546 may be deployed separately and alongside the balloon assembly 518. For example, the inflation lumen 510 be within a second catheter separate and apart from catheter 502. The second catheter may be deployed adjacent to catheter 502 such that the second catheter extends through the channel formed between a restrained portion 540 of the balloon assembly 518 and the vessel. In this way, the blood-flow barrier 546 is configured to receive the inflation fluid from the inflation lumen 510 separate and independently from the balloon assembly 518. The blood-flow barrier 546 may also be other similar blocking structures, deployed either in conjunction with or alongside the balloon assembly 518. For example, the blood-flow barrier 546 may be an occluder device (e.g., a PFO occluder, a cardioform septal occluder, a double-disk occluder, etc.), an umbrella-shaped blocking structure, or other similar structures that expand to block the vessel distal to the balloon assembly 518. For structures that do not require an inflation fluid to enter an expanded state, the blood-flow barrier 546 can be attached to a wire, catheter, or expansion lumen configured to deploy the blood-flow barrier 546.

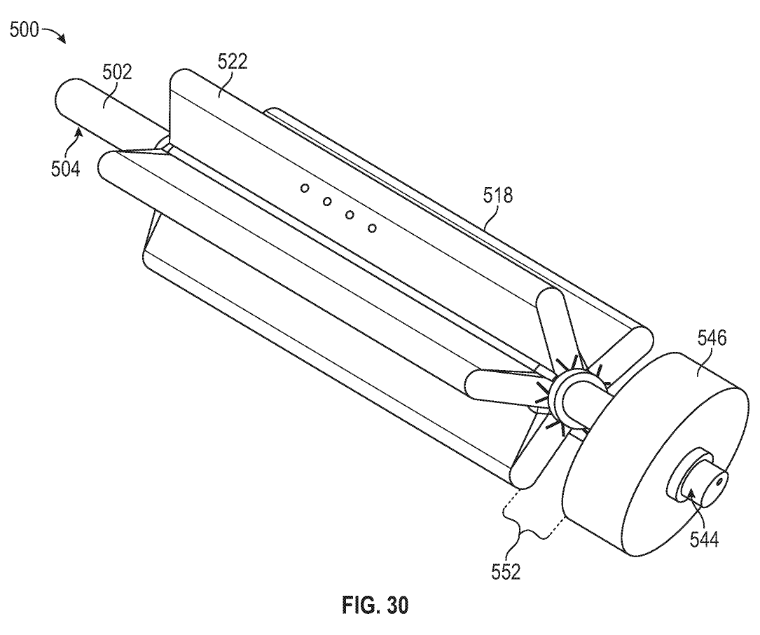

FIG. 30 shows an embodiment of the balloon catheter device 500 where the first outer surface 504 is contiguous with the second outer surface 544. In such embodiments, the blood-flow barrier 546 and the balloon assembly 518 are a fixed distance 552 (“X” mm, µm, etc.) apart. In other words, the blood-flow barrier 546 is configured to enter an expanded state (e.g., by receiving the inflation fluid from the inflation lumen 510) at the same or at a different time than the balloon 522 enters an expanded state by receiving the drug from the drug lumen 514. However, the blood-flow barrier 546 cannot move closer to or farther from the balloon assembly 518 because they are connected to contiguous outer surfaces (e.g., the first outer surface 504 and the second outer surface 544 as shown in FIG. 30).

### V. Construction of Example Embodiments

It should be noted that the term “example” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Additionally, it should be understood that features from one embodiment disclosed herein may be combined with features of other embodiments disclosed herein as one of ordinary skill in the art would understand. Other substitutions, modifications, changes and omissions may also be made in the design, operating conditions and arrangement of the various exemplary embodiments without departing from the scope of the present disclosure.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

As utilized herein, the terms “substantially,” “generally,” “approximately,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

What is claimed is:

1. A balloon catheter device comprising:
a catheter defining:
a guidewire lumen, and
a drug lumen; and
a balloon assembly coupled to a distal portion of the catheter, the balloon assembly comprising:
a balloon having a proximal end and a distal end that are coupled to an outer surface of the catheter, the balloon configured to receive a drug from the drug lumen and having an outer surface through which a plurality of openings extend,
a proximal retainer coupled to the outer surface of the catheter at the proximal end of the balloon,
a distal retainer coupled to the outer surface of the catheter at the distal end of the balloon, and
a plurality of restraints coupled between the proximal retainer and the distal retainer, each of the restraints extending along the balloon;
wherein, when the balloon is in an expanded state, the balloon has a plurality of restrained portions, each extending along a length of one of the restraints, and a plurality of radially projecting portions, each positioned between adjacent of the restrained portions, the radially projecting portions extending further radially outward from the catheter than the restrained portions.

2. The balloon catheter device of claim 1, wherein:
the distal retainer comprises a plurality of first arc-shaped portions, each of the first arc-shaped portions having a first arc length defined by adjacent of the restraints;
the proximal retainer comprises a plurality of second arc-shaped portions, each of the second arc-shaped portions having a second arc length defined by adjacent of the restraints; and
the first arc length is equal to the second arc length.

3. The balloon catheter device of claim 2, wherein the first arc-shaped portions of the distal retainer are circumferentially offset from the second arc-shaped portions of the proximal retainer.

4. The balloon catheter device of claim 1, further comprising:
a second catheter; and
a blood-flow barrier deployable via the second catheter and configured to expand to block a vessel.

5. The balloon catheter device of claim 4, wherein:
the second catheter defines an inflation lumen positioned within the catheter of the balloon catheter device; and
the blood-flow barrier has a proximal end and a distal end that are coupled to an outer surface of the second catheter, the blood-flow barrier configured to receive an inflation fluid from the inflation lumen.

6. The balloon catheter device of claim 4, wherein the blood-flow barrier comprises an occluder device.

7. The balloon catheter device of claim 1, wherein the balloon includes the restraints, the restraints formed from first portions of the balloon that are thicker than second portions of the balloon forming the radially projecting portions.

8. The balloon catheter device of claim 1, wherein the balloon includes the restraints, the restraints formed from at least one of portions of the balloon bonded together or portions of the balloon bonded to the catheter.

9. The balloon catheter device of claim 1, wherein the balloon assembly includes an external sheath that fits over the balloon, the external sheath including the restraints.

10. A balloon catheter device comprising:
a catheter defining:
a guidewire lumen, and
a drug lumen; and
a balloon assembly coupled to a distal portion of the catheter, the balloon assembly comprising:
a balloon having a proximal end and a distal end that are coupled to an outer surface of the catheter, the balloon configured to receive a drug from the drug lumen and having an outer surface through which a plurality of openings extend, and
a plurality of restraints extending between the proximal end of the balloon and an intermediate location between the proximal end and the distal end of the balloon, each of the restraints extending along the balloon;
wherein, when the balloon is in an expanded state, the balloon has a first region between the proximal end and the intermediate location, the first region including a plurality of restrained portions, each extending along a length of one of the restraints, and a plurality of radially projecting portions, each extending between adjacent of the restrained portions, the radially projecting portions extending further radially outward from the catheter than the restrained portions; and
wherein, when the balloon is in the expanded state, the balloon has a second region distal to the first region and configured to block a vessel.

11. The balloon catheter device of claim 10, wherein the balloon includes the restraints, the restraints formed from first portions of the balloon that are thicker than second portions of the balloon forming the radially projecting portions.

12. The balloon catheter device of claim 10, wherein the balloon includes the restraints, the restraints formed from at least one of portions of the balloon bonded together or portions of the balloon bonded to the catheter.

13. The balloon catheter device of claim 10, wherein the balloon assembly includes an external sheath that fits over the balloon, the external sheath including the restraints.

14. The balloon catheter device of claim 10, wherein:
the balloon assembly includes:
a proximal retainer coupled to the outer surface of the catheter at the proximal end of the balloon, and
a second retainer coupled to the outer surface of the catheter at the intermediate location; and
the restraints are coupled between the proximal retainer and the second retainer.

15. The balloon catheter device of claim 14, wherein:
the proximal retainer comprises a plurality of first arc-shaped portions defined by adjacent of the restraints;
the second retainer comprises a plurality of second arc-shaped portions defined by adjacent of the restraints; and
the first arc-shaped portions of the proximal retainer are circumferentially offset from the second arc-shaped portions of the second retainer.

16. A balloon catheter device comprising:
a catheter defining:
a guidewire lumen,
a drug lumen, and
an inflation lumen;
a balloon assembly coupled to a distal portion of the catheter, the balloon assembly comprising a balloon having a proximal end and a distal end that are coupled to a first outer surface of the catheter, the balloon configured to receive a drug from the drug lumen and having an outer surface through which a plurality of openings extend; and
a blood-flow barrier having a proximal end and a distal end that are coupled to a second outer surface of the catheter, the second outer surface distal to the first outer surface, the blood-flow barrier configured to receive an inflation fluid from the inflation lumen;
wherein, when the balloon is in an expanded state, the balloon has a plurality of restrained portions and a plurality of radially projecting portions, each positioned between adjacent of the restrained portions, the radially projecting portions extending further radially outward from the catheter than the restrained portions; and
wherein, when the blood-flow barrier is in an expanded state, the blood-flow barrier is configured to block a vessel.

17. The balloon catheter device of claim 16, wherein the first outer surface is contiguous with the second outer surface.

18. The balloon catheter device of claim 16, wherein the balloon includes a plurality of restraints extending between the proximal end of the balloon and the distal end of the balloon, each of the restraints extending along one of the restrained portions of the balloon to restrain the restrained portions of the balloon, the restraints formed from first portions of the balloon that are thicker than second portions of the balloon forming the radially projecting portions.

19. The balloon catheter device of claim 16, wherein the balloon includes a plurality of restraints extending between the proximal end of the balloon and the distal end of the balloon, each of the restraints extending along one of the restrained portions of the balloon to restrain the restrained portions of the balloon, the restraints formed from at least one of portions of the balloon bonded together or portions of the balloon bonded to the catheter.

20. The balloon catheter device of claim 16, wherein the balloon assembly includes an external sheath that fits over the balloon, the external sheath including a plurality of restraints extending between the proximal end of the balloon and the distal end of the balloon, each of the restraints extending along one of the restrained portions of the balloon to restrain the restrained portions of the balloon.
